(19) United States
(12) Patent Application Publication
(10) Pub. No.: US 2015/0216487 A1
(43) Pub. Date: Aug. 6, 2015

(54) Medical Apparatus

(71) Applicants: Hanno Dotzel, Langensendelbach (DE); Anjaü Jäger, Fürth (DE); Robert Kagermeier, Nürnberg (DE)

(72) Inventors: Hanno Dotzel, Langensendelbach (DE); Anjaü Jäger, Fürth (DE); Robert Kagermeier, Nürnberg (DE)

(21) Appl. No.: 14/607,228

(22) Filed: Jan. 28, 2015

(30) Foreign Application Priority Data
Feb. 5, 2014 (DE) 102014202024.7

(51) Int. Cl. A61B 6/04; A61B 8/00; A61B 19/00; A61G 7/08; A61G 13/10; A61B 5/055; A61B 5/00

(52) U.S. Cl. CPC A61B 6/0457; A61B 5/0555; A61B 6/0407; A61B 8/40; A61B 5/704; A61G 7/08; A61G 13/104; A61B 19/46; A61B 2019/464

(57) ABSTRACT

To enable simplified movement and/or control of a medical apparatus, the medical apparatus includes a traversing mechanism and at least one motor. The at least one motor is embodied to drive the traversing mechanism. The medical apparatus is characterized by at least one force sensor and a control apparatus. The at least one force sensor detects a force acting from the outside on the medical apparatus, and the control apparatus activates the at least one motor in dependence on the force detected by the at least one force sensor.

### RELATED CASE

This application claims the benefit of DE 102014202024.7, filed on Feb. 5, 2014, which is hereby incorporated by reference in its entirety.

### BACKGROUND

The present embodiments relate to a medical apparatus and a medical imaging device with a medical apparatus.

In the medical environment, in addition to stationary medical apparatuses, increasing use is being made of mobile medical apparatuses. These mobile medical apparatuses are typically embodied as movable and/or transportable. Mobile medical apparatuses of this kind may be used as required in different locations or, when not in use, be temporarily removed from their working environment and parked in a suitable location.

For example, for clinical purposes, in particular for transportation or for an examination by a medical imaging device, patients may be positioned on a medical apparatus embodied as a patient bearing apparatus, in particular a bearing surface of the medical apparatus. These patient bearing apparatuses may be embodied as mobile, i.e. movable and/or transportable.

Mobile medical apparatuses, for example mobile patient beds or mobile patient bearing apparatuses for medical imaging devices, are frequently difficult for an operator to move and/or maneuver. A large amount of effort may be required. Therefore, rollers that are as smooth-running as possible are used in the traversing mechanism of the medical apparatus. It is also possible to use a motor to drive the traversing mechanism of the medical apparatus.

### SUMMARY AND DETAILED DESCRIPTION

The scope of the present invention is defined solely by the appended claims and is not affected to any degree by the statements within this summary.

The present embodiments are based on the object of enabling simplified movement and/or control of a medical apparatus.

A medical apparatus has a traversing mechanism and at least one motor. The at least one motor is embodied to drive the traversing mechanism. The medical apparatus is characterized by at least one force sensor and a control apparatus. The at least one force sensor detects a force acting from the outside on the medical apparatus, and the control apparatus activates the at least one motor in dependence on the force detected by the at least one force sensor.

Here, the medical apparatus is in particular embodied as a mobile medical apparatus. The traversing mechanism may comprise at least one traversing device, for example at least one roller that is as smooth-running as possible. The at least one traversing device of the traversing mechanism is then advantageously driven by the motor. Here, the motor typically generates a driving torque, which is transmitted onto the traversing mechanism. Here, the motor may be an electric motor. The medical apparatus typically includes an energy storage unit, which, for example, supplies the motor and/or the force sensor with energy. In particular, the motor may assist an operator who moves, for example pushes and/or pulls, the medical apparatus.

To this end, the force sensor is embodied to detect the force acting from the outside on the medical apparatus. In particular, the force is exerted by the operator, such as when moving the medical apparatus. The force may, for example, be a push or pull exerted by the operator on the medical apparatus. The force may act on a guide apparatus, which enables an operator to move the medical apparatus. The force may also act on the traversing mechanism of the medical apparatus. Correspondingly, the force sensor may be advantageously positioned to detect the force.

The control apparatus may register the force detected by the force sensor and may then, in dependence on the force detected by the force sensor, activate the motor. The motor then drives the traversing mechanism. To this end, the control apparatus may be implemented in the medical apparatus. Advantageously, a logic, in particular a computer-controlled logic, may be implemented on the control apparatus, which determines whether a force was detected by the force sensor and/or whether the force detected exceeds a threshold value. The motor is advantageously only activated if a force was detected by the force sensor (e.g., if the medical apparatus is actually being pushed and/or pulled by the operator). The motor may then advantageously also be deactivated again when the force sensor no longer detects a force.

Hence, the control apparatus and the force sensor enable intelligent activation of the motor of the medical apparatus in dependence on whether the medical apparatus is actually to be moved by an operator. Therefore, the motor may assist the operator in a particularly suitable way with the movement of the medical apparatus. This enables the operator to move and/or maneuver the medical apparatus in a particularly simple way. Hence, this may relieve the stress on the operator, who is able to move the medical apparatus with a greatly reduced amount of force. Hence, fewer operators are required to move the medical apparatus. This in turn results in increased efficiency of the procedures in a clinical facility since the medical apparatus may be transported more simply and quickly to the respective destination.

One embodiment envisages a guide apparatus that enables an operator to move the medical apparatus. At least one force sensor is arranged on the guide apparatus. The guide apparatus may, for example, be arranged on the head side and/or foot side of the medical apparatus. Here, the guide apparatus typically enables the guidance of the movement of the medical apparatus by the operator. To this end, the guide apparatus may, for example, be embodied as a handle for the operator. Since the force sensor is arranged on the guide apparatus, direct transmission of the force acting on the guide apparatus to the force sensor is possible.

One embodiment envisages that the guide apparatus includes an elastic region. The at least one force sensor is arranged on the elastic region. The elastic region of the guide apparatus may be macroscopically deformed by the action of the force, for example by pressure or pulling by the operator. To this end, the elastic region is advantageously made of a deformable material, for example a spring material, rubber, foam or elastomer. The force sensor may be a mechanical switch, a pressure-sensitive coating of the elastic region, a capacitive recognition of a hand of an operator, and/or further sensors that appear advisable to the person skilled in the art. Advantageously, these elements are arranged on the guide apparatus and are able to register deformation of the elastic region of the guide apparatus. Hence, the guide apparatus simultaneously enables a particularly simple movement of the medical apparatus by the operator and an advantageous recognition of the force acting on the medical apparatus.

One embodiment envisages that the at least one motor is embodied to drive the traversing mechanism in at least two spatial directions. The at least one motor is activated in dependence on the force measured by the at least one force sensor along the at least two spatial directions. In particular, the force sensor is then embodied to detect the force acting on the medical apparatus along the at least two spatial directions. This enables the operator to maneuver the medical apparatus particularly intuitively.

One embodiment envisages that the control apparatus sets a power of the at least one motor in dependence on the force detected by the at least one force sensor. Advantageously, a higher force measured by the force sensor results in a higher power of the motor. If the force acting on the medical apparatus drops, the control apparatus advantageously also reduces the power of the motor. Hence, the motor may be optimally driven with optimum adaptation to the respective application of the traversing mechanism. If, for example, only a slow movement of the medical apparatus is desired, the motor of the traversing mechanism is also only driven with low power. Hence, the power of the motor may advantageously be adapted to the operating state.

One embodiment envisages a setting element by which a power of the at least one motor may be set. In particular, the operator is enabled to make a setting of the power of the at least one motor. The setting element may enable a setting, to which part of the power detected by the force sensor, in particular applied by the operator, is additionally connected by the motor. It is also possible for the absolute power of the motor to be set by the setting element. The setting element may be embodied as a physical (e.g., arranged on a guide apparatus) switch and/or a sliding control. The setting element may also be embodied as a part of an operator console arranged on the medical apparatus. It is also possible for further setting elements that appear advantageous to the person skilled in the art to be implemented. Hence, the setting element enables an individual setting the power of the motor in accordance with the operator's wishes.

One embodiment envisages a feedback apparatus that is embodied to forward information on an activation status of the at least one motor to an operator. Advantageously, the feedback apparatus is arranged on the guide apparatus. Hence, the feedback apparatus may provide the operator with haptic feedback by the guide apparatus. The feedback apparatus may, for example, cause a vibration of the guide apparatus, such as vibration of the elastic region of the guide apparatus. It is also possible for another feedback apparatus that appears advantageous to the person skilled in the art to be implemented, for example an optical display. The feedback apparatus then advantageously confirms to the operator that the motor was activated due to the force detected by the force sensor. Hence, the operator is able to establish the activation status of the motor in a particularly simple way.

One embodiment envisages a switch which is embodied to switch off the at least one motor. Hence, it is possible for the operator to switch of the motor manually so that the medical apparatus may again be moved in the conventional way without the assistance of the motor. Advantageously, the switch is embodied as an emergency stop switch, such as a clearly marked emergency stop switch. Hence, the motor may be advantageously switched off immediately if, for example, there is a risk of the medical apparatus colliding with an obstacle. Hence, the safety of people or objects in the route of the medical apparatus may be increased.

One embodiment envisages an authentication apparatus. The at least one motor is activated in dependence on an authentication of an operator by the authentication apparatus. To this end, the authentication apparatus is advantageously connected to the control apparatus for the purpose of data exchange, that is, in particular via a data exchange unit. The authentication apparatus may, for authentication of the operator, for example, use an identification by radio waves and/or near-field communication. To this end, the operator may have corresponding communication devices. Obviously, other possibilities for the authentication of the operator that appear advantageous to the person skilled in the art are conceivable. Hence, the safety of the medical apparatus may be increased since the motor of the medical apparatus may only be activated by authenticated people.

One embodiment envisages that the medical apparatus is embodied as a patient bearing apparatus including a bearing surface for bearing a patient. The patient bearing apparatus is hence embodied as mobile. The movement of the patient bearing apparatus is assisted by at least one motor, which is activated in dependence on a force detected by at least one force sensor. This makes particularly smooth-running and simple movement of the patient bearing apparatus possible for an operator even if, for example, heavy patients are positioned on the patient bearing apparatus. Hence, patients may be transported more quickly and simply to their destinations in a clinical facility, for example an examination room. The force sensor is advantageously embodied as a three-dimensional force sensor in order to register a movement of the guide apparatus by the operator upward or downward along the weight force of the patient bearing apparatus. The control apparatus may then, with reference to this movement, activate a further motor, which enables a vertical movement perpendicular to the plane of travel of the patient bearing apparatus (e.g., a raising or lowering) of the bearing surface of the patient bearing apparatus. It is possible, for example, to achieve a simplified positioning of the patient on the patient bearing apparatus.

One embodiment envisages that the switch is arranged on the bearing surface. The switch on the bearing surface is an emergency stop switch. In particular, the switch is arranged on the bearing surface such that the switch may be reached by a patient positioned on the bearing surface. Here, a patient positioned on the bearing surface is typically positioned lying on their back with the head pointing in the direction of the head end of the patient bearing apparatus. This positioning of the switch enables the motor to be switched off by the patient, for example in a hazardous situation or when the operator is distracted. Hence, it is in turn possible to increase the safety of the patient or the safety of people or objects in the route of the patient bearing apparatus.

The present embodiments are also based on a medical imaging device including a medical apparatus embodied as a patient bearing apparatus. In particular, the patient bearing apparatus is matched to the medical imaging device such that a patient positioned on the patient bearing apparatus may be examined by the medical imaging device. Here, the patient bearing apparatus enables particularly simple transportation of the patient to the medical imaging device. Further, a particularly simple sequence of operations is enabled since the patient does not have to be transferred from a transport apparatus to a patient table in the medical imaging device.

One embodiment of the medical imaging device envisages a docking apparatus for the medical apparatus. In particular, the docking is performed at a reduced speed of the patient bearing apparatus, so that reliable docking of the patient bearing apparatus is ensured. Low power of the motor is used during docking. The automatic docking of the medical apparatus on the medical imaging device results in simplification of the sequence of operations during an examination of the patient by the medical imaging device.

One embodiment of the medical imaging device envisages an operating element, wherein an actuation of the operating element by an operator triggers automatic docking of the medical apparatus on the docking apparatus. Here, the operating element may be arranged on the patient bearing apparatus and/or on the medical imaging device.

### DESCRIPTION OF THE DRAWINGS

FIG. 1 a schematic diagram of a medical apparatus embodied as a patient bearing apparatus, and

FIG. 2 a medical imaging device with a medical apparatus embodied as a patient bearing apparatus in a schematic diagram.

### DETAILED DESCRIPTION OF THE PREFERRED EMBODIMENTS

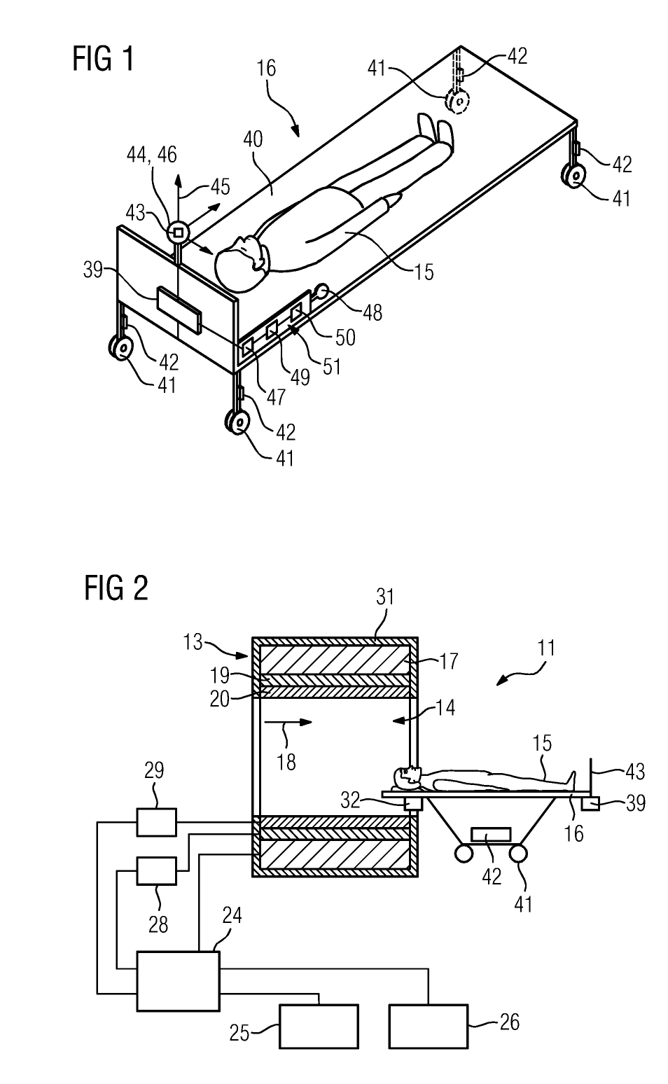

FIG. 1 shows a schematic diagram of a medical apparatus 16. In the case shown, the medical apparatus 16 is embodied as a mobile patient bearing apparatus 16 for a medical imaging device 11 (see FIG. 2). To this end, the patient bearing apparatus includes a bearing surface 40 for a patient 15.

Alternatively, the medical apparatus 16 shown may also be a mobile patient bearing apparatus 16 for an interventional examination apparatus and/or a therapeutic apparatus, for example an angiography, cardiology, nephrology or urology examination apparatus. Alternatively, the medical apparatus 16 shown may also be a mobile operating table and/or a mobile sickbed.

Mobile medical apparatuses that do not have any bearing surface 40 for a patient 15 are also conceivable. For example, the medical apparatus may also be a mobile gantry for a medical imaging device. Alternatively, the medical apparatus may be a mobile X-ray system for radiography, fluoroscopy or mammography. The medical apparatus may also be embodied as a mobile C-arm X-ray system for a surgical, angiographic, or cardiological application. It is also conceivable for the medical apparatus to be a mobile assessment station and/or operator station for medical personnel. It is also possible for the medical apparatus to be embodied as a mobile device for intensive care, for example as a mobile monitoring device, respirator, infusion device, and/or dialyzer. Finally, the medical apparatus may also be a mobile robot system for medical applications.

The medical apparatus 16 shown in FIG. 1 is formed by a mobile patient bearing apparatus 16 designed to transport a patient 15, for example from a sick room to a medical imaging device 11. The patient bearing apparatus 16 also includes a coupling unit (not shown in any further detail) for docking with the medical imaging device 11 (see FIG. 2).

The patient bearing apparatus 16 includes a bearing surface 40 on which a patient 15 is borne. The patient bearing apparatus 16 also includes a traversing mechanism 41. In the case shown, the traversing mechanism 41 is four rollers. The patient bearing apparatus 16 also includes a motor 42 to drive the traversing mechanism 41, in the case shown, four individual motors 42 each to drive one of the four rollers 41. The motors 42 are embodied to drive the traversing mechanism 41 in two spatial directions on the plane of travel of the patient bearing apparatus 16. The four motors 42 are also embodied to move the bearing surface 40 in the vertical direction (i.e., to raise and lower the bearing surface 40). A different number of motors 42 is also possible. For example, it is possible for there to be only two motors 42, which then, for example, drive two of the four rollers 41. There may also be a different number of rollers 41.

The patient bearing apparatus 16 further includes a force sensor 43 and a control apparatus 39. The force sensor 43 detects force acting from the outside on the patient bearing apparatus 16, and the control apparatus 39 activates the motors 42 in dependence on the force detected by the force sensor 43. To this end, the force sensor 43 is connected to the control apparatus 39 for the purpose of data exchange. The control apparatus 39 is connected to the four motors 42 for the purpose of data exchange. The control apparatus 39 may set the power of the motors 42 in dependence on the force detected by the force sensor 43.

The patient bearing apparatus 16 includes a guide apparatus 44, which enables the operator to move the patient bearing apparatus 16. In the case shown, the guide apparatus 44 is embodied as a handle 44. The operator is not shown explicitly. Only the movement 45 of the guide apparatus 44 performed by the operator in three spatial directions to control the movement of the patient bearing apparatus 16 is shown. The guide apparatus 44 includes an elastic region on which the force sensor 43 is arranged. In the case shown, the handle 44 is provided with a pressure-sensitive coating, which transfers the pressure or traction exerted by the operator on the handle 44 in the three spatial directions onto the force sensor 43. The force sensor 43 may detect and/or measure the force exerted by the operator on the handle 44.

The motors 42 are then activated by the control apparatus 39 in dependence on the force measured by the force sensor 43 along the three spatial directions. If, for example, the operator moves the handle 44 in a horizontal direction, such as perpendicular to a weight force of the patient bearing apparatus 16, the control apparatus 39 causes the motors 42 to drive the traversing mechanism 41 of the patient bearing apparatus 16 in dependence on the force detected by the force sensor 43. The patient bearing apparatus 16 moves in a horizontal movement on the plane of travel or executes a curve. To this end, the motors 42 generate different driving torques, which drives the rollers 41 at different speeds resulting in a curved path of the patient bearing apparatus 16. If, for example, the operator moves the handle 44 vertically upward (e.g., opposite to the weight force acting on the patient bearing apparatus 16), in dependence on the force detected by the force sensor 43, the control apparatus 39 causes the motors 42 to move the bearing surface 40 of the patient bearing apparatus 16 vertically upward.

Simultaneously, the patient bearing apparatus 16 includes a feedback apparatus 46 embodied to forward information on the activation status of the motors 42 to the operator. In the case shown, the feedback apparatus 46 is implemented in the handle 44. The handle 44 includes a vibration motor, which causes the handle 44 to vibrate when the motors 42 are activated and hence provides feedback for the operator on the activation status of the motors. Here, the vibration motor is controlled by the control apparatus 39. The control apparatus 39 detects the activation status the motors 42. The feedback apparatus 46 is connected to with the control apparatus 39 or the motors 42 for the purpose of data exchange.

The patient bearing apparatus 16 also includes an operator console 51 for the operator 45. The operator console 51 includes a setting element 47, a switch 48, an authentication apparatus 49, and an operating element 50.

Here, the setting element 47 enables the operator to set the power of the motors 42. To this end, the setting element 47 is connected to the motors 42 via the control apparatus 39 for the purpose of data exchange. The control apparatus 39 sets the power of the motors 42 in dependence on the setting of the setting element 47 registered by the control apparatus 39.

The switch 48 is embodied to switch off the motors 42. To this end, the switch 48 is connected with the motors 42 via the control apparatus 39 for the purpose of data exchange. The control apparatus 39 deactivates the motors 42 when the control apparatus 39 registers an actuation of the switch 48. The switch 48 is arranged on the bearing surface 40 so that the switch 48 may be reached by a patient 15 positioned on the bearing surface 40. The switch 48 is also clearly identified as an emergency stop switch.

The authentication apparatus 49 enables an authentication of the operator 45. The motors 42 are activated in dependence on the authentication of the operator by the authentication apparatus 49. To this end, the authentication apparatus 49 is connected to the motors 42 via the control apparatus 39 for the purpose of data exchange. Here, the control apparatus 39 checks the authentication of the operator by the authentication apparatus 49 and, in the event of successful authentication of the operator, activates the motors 42.

The operating element 50 may be actuated by the operator. The actuation of the operating element 50 by the operator triggers automatic docking of the patient bearing apparatus 16 to a docking apparatus 32 (see FIG. 2) of the medical imaging device 11. Here, the automatic docking of the patient bearing apparatus 16 is executed by a slow movement of the traversing mechanism 41 (i.e., a lower power 42 of the motors 42). The automatic docking of the patient bearing apparatus 16 is also controlled by the control apparatus 39.

As already explained, the control apparatus 39 of the patient bearing apparatus 16 activates the motors 42 centrally in dependence on the force detected by the force sensor 43 and/or on entries made by the operator in the operator console 51. To this end, the control apparatus 39 includes the necessary logic, in particular software and/or computer programs, stored in a memory unit of the control apparatus 39.

FIG. 2 shows a medical imaging device 11 with a medical apparatus 16 embodied as a patient bearing apparatus 16 in a schematic diagram. The medical imaging device 11 is embodied by way of example as a magnetic imaging device 11. The medical imaging device 11 may alternatively also be a single photon emission computed tomography device (SPECT device), a positron emission tomography (PET-device), a computed tomography device, an ultrasound device, an X-ray device, or a C-arm device. Combined medical imaging devices 11 are also possible in any combination of several of the named imaging modalities.

The magnetic imaging device 11 includes a detector unit formed from a magnetic unit 13 with a main magnet 17 to generate a strong and in particular constant main magnetic field 18. The magnetic imaging device 11 also includes a cylindrical patient receiving region 14 to receive a patient 15. The patient receiving region 14 is enclosed in a circumferential direction by the magnetic unit 13 in a cylindrical shape. The magnetic unit 13 is screened from the outside by a lining of the housing 31 of the magnetic imaging device.

The magnetic unit 13 further comprises a gradient coil unit 19 to generate magnetic field gradients, which are used for spatial encoding during imaging. The gradient coil unit 19 is controlled by a gradient control unit 28. The magnetic unit 13 also includes a high-frequency antenna unit 20, which in the case shown, is embodied as a fixed integral body coil in the magnetic imaging device 10, and a high-frequency antenna control unit 29 to excite a polarization, which is established in the main magnetic field 18 generated by the main magnet 17. The high-frequency antenna unit 20 is controlled by the high-frequency antenna control unit 29 and radiates high-frequency magnetic resonance sequences into an examination chamber, substantially formed by the patient receiving region 14. The high-frequency antenna unit 20 is also embodied to receive magnetic resonance signals from the patient 15.

To control the main magnet 17, the gradient control unit 28 and the high-frequency antenna control unit 29, the magnetic imaging device 11 includes a computer unit 24. The computer unit 24 centrally controls the magnetic imaging device 11, such as, for example, the performance of a predetermined imaging gradient echo sequence. Control information such as, for example, imaging parameters, and reconstructed magnetic resonance images may be displayed on a display unit 25, for example on at least one monitor, of the magnetic imaging device 11 to a user. The magnetic imaging device 11 also includes an input unit 26 by which information and/or parameters may be input by a user during a measuring process. The computer unit 24 may include the gradient control unit 28 and/or high-frequency antenna control unit 29.

The magnetic imaging device 11 depicted may include further components, such as those normally found in magnetic resonance devices 11. In addition, the general mode of operation of a magnetic imaging device 11 is known to the person skilled in the art so there will be no detailed description of the further components.

The magnetic imaging device 11 depicted further includes a medical apparatus 16 embodied as a patient bearing apparatus 16. The patient 15 may be pushed by the patient bearing apparatus 16 of the magnetic imaging device 11 into the patient receiving region 14. Here, the bearing surface 40 of the patient bearing apparatus 16 is arranged movably inside the magnetic imaging device 11.

The patient bearing apparatus 16 is embodied as compatible with magnet-resonance. To this end, the patient bearing apparatus 16 is mainly and/or preferably almost completely made of non-magnetic materials. The use of non-magnetic materials reduces or excludes any influence on the magnetic fields or high-frequency waves in the magnetic imaging device 11. The use of non-magnetic materials also prevents artifacts forming on the images recorded by the magnetic imaging device 11.

The magnetic imaging device 11 also includes a docking apparatus 32 for the patient bearing apparatus 16. Actuation of the operating element 50 of the patient bearing apparatus 16 by the operator triggers automatic docking of the patient bearing apparatus 16 on the docking apparatus 32. The patient bearing apparatus 16 may, for example after the end of the examination of the patient 15 in the magnetic imaging device 11, be undocked again from the docking apparatus 32 of the magnetic imaging device 11 so that the patient 15 may be transported away from the magnetic imaging device 11.

Although the invention was illustrated and described in detail by the preferred exemplary embodiments, the invention is not restricted by the disclosed examples and other variations may be derived herefrom by the person skilled in the art without departing from the scope of protection of the invention.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A medical apparatus comprising:
a traversing mechanism;
at least one motor configured to drive the traversing mechanism;
at least one force sensor; and
a control apparatus configured toactivate the at least one motor in dependence on a force acting on an outside of the medical apparatus detected by the at least one force sensor.

2. The medical apparatus as claimed in claim 1, further comprising a guide apparatus configured to provide for an operator to move the medical apparatus, wherein the at least one force sensor being arranged on the guide apparatus.

3. The medical apparatus as claimed in claim 2, wherein the guide apparatus comprises an elastic region, and wherein the at least one force sensor is arranged on the elastic region.

4. The medical apparatus as claimed in claim 1, wherein the at least one motor is configured to drive the traversing mechanism in at least two spatial directions, wherein the at least one motor is activated in dependence on the force measured by the at least one force sensor along the at least two spatial directions.

5. The medical apparatus as claimed in claim 1, wherein the control apparatus sets a power of the at least one motor in dependence on the force detected by the at least one force sensor.

6. The medical apparatus as claimed in claim 1, further comprising a setting element configured to set a power of the at least one motor.

7. The medical apparatus as claimed in claim 1, further comprising a feedback apparatus configured to forward information on an activation status of the at least one motor to an operator.

8. The medical apparatus as claimed in claim 1, further comprising a switch configured to switch off the at least one motor.

9. The medical apparatus as claimed in claim 1, further comprising an authentication apparatus, wherein the at least one motor is activated in dependence on an authentication of an operator by the authentication apparatus.

10. The medical apparatus as claimed in claim 1, further comprising a bearing surface for bearing a patient.

11. The medical apparatus as claimed in claim 8, further comprising a bearing surface for bearing a patient, wherein the switch is arranged on the bearing surface.

12. The medical apparatus as claimed in claim 3, wherein the at least one motor is configured to drive the traversing mechanism in at least two spatial directions, wherein the at least one motor is activated in dependence on the force measured by the at least one force sensor along the at least two spatial directions.

13. The medical apparatus as claimed in claim 12, wherein the control apparatus sets a power of the at least one motor in dependence on the force detected by the at least one force sensor.

14. The medical apparatus as claimed in claim 13, further comprising a switch configured to switch off the at least one motor.

15. The medical apparatus as claimed in claim 14, further comprising an authentication apparatus, wherein the at least one motor is activated in dependence on an authentication of an operator by the authentication apparatus.

16. The medical apparatus as claimed in claim 15, further comprising a bearing surface for bearing a patient, wherein the switch is arranged on the bearing surface.

17. A medical imaging device comprising:
a medical apparatus comprising:
a traversing mechanism;
at least one motor configured to drive the traversing mechanism;
at least one force sensor;
a control apparatus configured toactivate the at least one motor in dependence on a force acting on an outside of the medical apparatus detected by the at least one force sensor; and
a bearing surface for bearing a patient.

18. The medical imaging device as claimed in claim 17, further comprising a docking apparatus for the medical apparatus.

19. The medical imaging apparatus as claimed in claim 18, further comprising an operating element, wherein an actuation of the operating element by an operator triggers automatic docking of the medical apparatus on the docking apparatus.
